(19) United States
(12) Patent Application Publication
(10) Pub. No.: US 2003/0212449 A1
(43) Pub. Date: Nov. 13, 2003

(54) Hybrid stent

(76) Inventor: Daniel L. Cox, Palo Alto, CA (US)

Correspondence Address: FULWIDER PATTON LEE & UTECHT, LLP, HOWARD HUGHES CENTER, 6060 CENTER DRIVE, TENTH FLOOR, LOS ANGELES, CA, 90045, US

(21) Appl. No.: 10/035,037

(22) Filed: Dec. 28, 2001

(51) Int. Cl. A61F 2/06

(52) U.S. Cl. 623/1.15

(57) ABSTRACT

The present invention is directed to an expandable hybrid stent for implantation in a body lumen, such as a coronary artery. The stent generally consists of metallic cylindrical rings used in connection with polymeric links, polymeric wire or a polymeric coil. The metallic cylindrical rings can include series of radially expandable cylindrical rings longitudinally aligned on a common axis of the stent. The rings can be interconnected by one or more polymeric links or can be disposed over an inner member consisting of a polymeric coil or a series of polymeric wires. Adjacent cylindrical rings can also be connected and arranged in a coil-like spiraling form without interconnecting links. The polymeric material forming the polymeric links, polymeric wire or the polymeric coil can provide longitudinal and flexural flexibility to the stent while maintaining sufficient column strength to space the cylindrical rings along the longitudinal axis. The metallic material forming the rings in either separate form or coil-like form can provide the necessary radial stiffness.

### BACKGROUND OF THE INVENTION

This invention relates to expandable endoprosthesis devices, generally called stents, which are adapted to be implanted into a patient's body lumen, such as a blood vessel, to maintain the patency thereof. These devices are useful in the treatment of atherosclerotic stenosis in blood vessels.

Stents are generally tubular-shaped devices which function to hold open a segment of a blood vessel, coronary artery, or other anatomical lumen. Various means have been described to deliver and implant stents. One method frequently described for delivering a stent to a desired intraluminal location includes mounting the expandable stent on an expandable member, such as a balloon, provided on the distal end of an intravascular catheter, advancing the catheter to the desired location within the patient's body lumen, inflating the balloon on the catheter to expand the stent into a permanent expanded condition and then deflating the balloon and removing the catheter. One of the difficulties encountered using prior stents involved maintaining the radial rigidity needed to hold open a body lumen while at the same time maintaining the longitudinal flexibility of the stent to facilitate its delivery. Once the stent is mounted on the balloon portion of the catheter, it is often delivered through tortuous vessels, including tortuous coronary arteries. The stent must have numerous properties and characteristics, including a high degree of flexibility in order to appropriately navigate the tortuous coronary arteries. This flexibility must be balanced against other features including radial strength once the stent has been expanded and implanted in the artery. While other numerous prior art stents have had sufficient radial strength to hold open and maintain the patency of a coronary artery, they have lacked the flexibility required to easily navigate tortuous vessels without damaging the vessels during delivery.

Generally speaking, most prior art intravascular stents are formed from a metal such as stainless steel, which is balloon expandable and plastically deforms upon expansion to hold open a vessel. The component parts of these types of stents typically are all formed of the same type of metal, i.e., stainless steel. Other types of prior art stents may be formed from a polymer, again all of the component parts being formed from the same polymer material. These types of stents, the ones formed from a metal and the ones formed from a polymer, each have advantages and disadvantages. One of the advantages of the metallic stents is their high radial strength once expanded and implanted in the vessel. A disadvantage may be that the metallic stent lacks flexibility which is important during the delivery of the stent to the target site. With respect to polymer stents, they may have a tendency to be quite flexible and are advantageous for use during delivery through tortuous vessels, however, such polymer stents may lack the radial strength necessary to adequately support the lumen once implanted.

What has been needed and heretofore unavailable is a stent which has a high degree of flexibility so that it can be advanced through tortuous passageways and can be readily expanded and yet have the mechanical strength to hold open the body lumen into which it expanded. The present invention satisfied this need.

### SUMMARY OF THE INVENTION

The present invention is directed to an expandable hybrid stent for implantation in a body lumen, such as a coronary artery. The stent generally consists of metallic cylindrical rings used in connection with polymeric links, polymeric wire or a polymeric coil. The metallic cylindrical rings can include series of radially expandable cylindrical rings longitudinally aligned on a common axis of the stent. The rings can be interconnected by one or more polymeric links or can be disposed over an inner member consisting of a polymeric coil or a series of polymeric wires. Adjacent cylindrical rings can also be connected and arranged in a coil-like spiraling form without interconnecting links. The polymeric material forming the polymeric links, polymeric wire or the polymeric coil can provide longitudinal and flexural flexibility to the stent while maintaining sufficient column strength to space the cylindrical rings along the longitudinal axis. The metallic material forming the rings in either separate form or coil-like form can provide the necessary radial stiffness.

The metallic rings of the stent can be connected in new ways compared to a single material metallic tubular stent. The ring connection point is not limited to being at specific ring locations. A spiral ring connection can result in a semi-covered stent, but with significant open area. Opposing spirals, one of which is polymeric and the other a helical metal ring can provide additional stent rigidity if required. The additional surface area of the polymer in spiral form can allow a bioactive coating to have a much greater surface area and therefore more activity than a coated stent. The same is true for a drug delivery stent using the stent configurations of the present invention.

The configuration of the stent of the present invention can also enhance stent retention. This is achieved by bonding the polymeric component to the inner surface of the metallic component. With this type of attachment the polymer can be in contact with the balloon utilized in a balloon expandable stent during stent delivery. The stent retention is achieved by selecting polymers with the desired hardness, frictional, and width values.

A low durometer polymer in this case can provide two benefits. First, many polymers are somewhat tacky and the friction between the polymer and balloon can help keep the stent on the balloon. Second, the mechanical compression of the polymer during and after stent crimping can provide a higher gripping force of the stent on the balloon. A low durometer polymer can be selectively and strategically placed on the stent to achieve this effect. The other ring connections can be a different polymer or durometer to achieve other stent properties.

The stent of the present invention can be particularly well suited to neurovascular applications where flexibility is extremely important to negotiate through the carotid siphon and into the middle cerebral artery. The tremendous flexibility of the polymer connector can greatly enhance the overall flexibility of the stent.

Another neurovascular application of this stent is for wide neck aneurysms. Because connections between the metallic rings can be polymeric, they can be designed specifically for this application and still obtain the flexibility needed to reach the anterior cerebral, middle cerebral or basilar artery. In this case the polymeric connectors could be made wide or narrow with closely spaced connectors in the region of the aneurysm neck. The metallic rings can also be spaced farther apart than for a typical coronary application to prevent occlusion of perforator vessels.

The stent of the present invention can generally include a series of metallic cylindrical rings and a polymeric coil. The cylindrical rings and polymeric coil are aligned along a longitudinal axis of the stent. The cylindrical rings are disposed around the polymeric coil to enhance the radial strength of the stent and spaced apart longitudinally to provide the stent with longitudinal flexibility. In the case of a balloon expandable catheter system the cylindrical rings and the polymeric coil remain closely coupled from the time the stent is crimped onto the delivery system to the time the stent is expanded and implanted into a body lumen. Accordingly, both the cylindrical rings and the polymeric coil have first delivery diameters in the crimped state and second implanted diameters.

The stent of the present invention can also generally include a polymeric coil and a metallic coil, the metallic coil consisting essentially of cylindrical rings arranged in a coil-like form. The metallic coil and polymeric coil are aligned along a longitudinal axis of the stent. The metallic coil is disposed around the polymeric coil to enhance the radial strength of the stent while also providing the stent with longitudinal flexibility. In the case of a balloon expandable catheter system the metallic coil and the polymeric coil remain closely coupled from the time the stent is crimped onto the delivery system to the time the stent is expanded and implanted into a body lumen. Accordingly, both the metallic coil and the polymeric coil have first delivery diameters in the crimped state and second implanted diameters.

The stent of the present invention can also generally include a series of metallic cylindrical rings and a series of polymeric wires. The cylindrical rings and wires are aligned along a longitudinal axis of the stent. The cylindrical rings are disposed around the wires to enhance the radial strength of the stent and spaced apart longitudinally to provide the stent with longitudinal flexibility. In the case of a balloon expandable catheter system the cylindrical rings and the wire remain closely coupled from the time the stent is crimped onto the delivery system to the time the stent is expanded and implanted into a body lumen. Accordingly, both the metallic cylindrical rings and the polymeric wires have first delivery diameters in the crimped state and second implanted diameters.

The metallic cylindrical rings whether separate or formed in a coil-like configuration can have undulations including peaks and valleys generally formed as U, W, and Y members. The peaks of each cylindrical ring can be axially aligned with the valleys of each adjacent cylindrical ring to provide the desired flexibility. The resulting stent structure is a series of radially expandable cylindrical rings which are spaced longitudinally close enough so that small dissections in the wall of a body lumen may be pressed back into position against the lumenal wall, but not so close as to compromise the longitudinal flexibility of the stent.

The metallic cylindrical rings can be placed over the polymeric coil or the polymeric wire and attached at desired locations. Methods of attachment can include slotting the contacting surfaces, applying a permanent bonding agent, and an interference fit between the metallic cylindrical rings and the polymeric coil. The rings can also be connected by polymeric links as mentioned above. The polymeric coil, polymeric wire, or polymeric links will provide flexibility and allow the stent to easily bend or flex along its longitudinal axis as the stent navigates through tortuous vessels or coronary arteries. The combination of the flexible metallic cylindrical rings when used in connection with either the polymeric coil, the polymeric wire, or the polymeric links cumulatively provide a stent which is flexible along its length and about its longitudinal axis, yet is still relatively stiff in the radial direction after it has been expanded in order to maintain the patency of a vessel and to resist collapse.

The stent embodying features of the invention can be readily delivered to the desired body lumen, such as a coronary artery (peripheral vessels, bile ducts, etc.), by mounting the stent on an expandable member of a delivery catheter, for example a balloon, and advancing the catheter and stent assembly through the body lumen to the target site. Generally, a crimping tool is used to crimp the stent onto the balloon portion of the catheter so that the stent does not move longitudinally relative to the balloon portion of the catheter during delivery through the arteries, and during expansion of the stent at the target site.

During the crimping process the metallic cylindrical rings undergo a plastic deformation and radially compress while the polymeric coil wire also radially compresses within the rings to removably secure the stent to the balloon.

After insertion of the stent to the desired location of delivery, the balloon is inflated to implant the stent. The metallic cylindrical rings of the stent are plastically deformed when expanded so that the stent will remain in the expanded condition and therefore they must be sufficiently rigid when expanded to prevent the collapse thereof in use. The cylindrical rings also can hold the polymeric coil or wire in the expanded state. During expansion of the stent, portions of the cylindrical rings may tip outwardly resulting in projecting members on the outer surface of the expanded stent. These projecting members tip radially outwardly from the outer surface of the stent and embed into the vessel wall and help secure the expanded stent so that it does not move once it is implanted. During this expansion the diameter of the cylindrical rings and the polymeric coil or wire increase at roughly the same rate. The similar rate of expansion helps keep the cylindrical rings and polymeric coil or wire closely coupled together.

It is to be recognized that the stent of the present invention can be self-expanding or balloon-expanded. Moreover, the present invention can be modified to be used in other body lumens including highly tortuous and distal vasculature as well as to create whole or portions of other medical devices or markers placed on such devices.

Other features and advantages of the present invention will become more apparent from the following detailed description of the invention when taken in conjunction with the accompanying exemplary drawings.

### BRIEF DESCRIPTION OF THE DRAWINGS

FIG. 1 is an elevational view, partially in section, of a stent embodying features of the invention including separate coils and a polymeric wire which is mounted on a delivery catheter and disposed within an artery.

FIG. 2 is an elevational view, partially in section, similar to that shown in FIG. 1 wherein the stent is expanded within an artery.

FIG. 3 is an elevational view, partially in section, depicting the expanded stent within the artery after withdrawal of the delivery catheter.

FIG. 4 is a perspective view of the stent in the crimped state.

FIG. 5 is a plan view of a flattened section of the stent of the invention, illustrating the cylindrical rings attached to the polymeric coil in the crimped state.

FIG. 6 is a perspective view of the stent of FIG. 4 after it is fully expanded depicting some portions of the stent projecting radially outwardly.

FIG. 7 is a perspective view of the stent in the crimped state.

FIG. 8 is a plan view of a flattened section of the stent of the invention, illustrating the cylindrical rings attached to the polymeric coil in the crimped state.

FIG. 9 is a perspective view of the stent of FIG. 6 after it is fully expanded depicting some portions of the stent projecting radially outwardly.

FIG. 10 is a perspective view of the stent in the crimped state.

FIG. 11 is a plan view of a flattened section of the stent of the invention, illustrating the cylindrical rings attached to the polymeric wire in the crimped state.

FIG. 12 is a perspective view of the stent of FIGS. 10 and 11 after it is fully expanded depicting some portions of the stent projecting radially outwardly.

FIG. 13 is a perspective view of the stent in the crimped state.

FIG. 14 is a plan view of a flattened section of the stent of the invention, illustrating the cylindrical rings attached to the polymeric wire in the crimped state.

FIG. 15 is a perspective view of the stent of FIGS. 13 and 14 after it is fully expanded depicting some portions of the stent projecting radially outwardly.

FIG. 16 is a perspective view of the stent in the crimped state.

FIG. 17 is a plan view of a flattened section of the stent of the invention, illustrating the cylindrical rings and differently sized polymeric links in the crimped state.

FIG. 18 is a perspective view of the stent of FIGS. 16 and 17 after it is fully expanded depicting some portions of the stent projecting radially outwardly.

FIG. 19 is a perspective view of the stent in the crimped state.

FIG. 20 is a plan view of a flattened section of the stent of the invention, illustrating the cylindrical rings and varied polymeric links in the crimped state.

FIG. 21 is a perspective view of the stent of FIGS. 19 and 20 after it is fully expanded depicting some portions of the stent projecting radially outwardly.

FIG. 22 is a perspective view of the stent in the crimped state.

FIG. 23 is a plan view of a flattened section of the stent of the invention, illustrating the cylindrical rings and varied polymeric links in the crimped state.

FIG. 24 is a perspective view of the stent of FIGS. 22 and 23 after it is fully expanded depicting some portions of the stent projecting radially outwardly.

FIG. 25 is a perspective view of the stent in the crimped state.

FIG. 26 is a plan view of a flattened section of the stent of the invention, illustrating the cylindrical rings and varied polymeric links in the crimped state.

FIG. 27 is a perspective view of the stent of FIGS. 25 and 26 after it is fully expanded depicting some portions of the stent projecting radially outwardly.

FIG. 28 is a perspective view of the stent in the crimped state.

FIG. 29 is a plan view of a flattened section of the stent of the invention, illustrating the cylindrical rings and varied polymeric links in the crimped state.

FIG. 30 is a perspective view of the stent of FIGS. 28 and 29 expanded depicting some portions of the stent projecting radially outwardly.

### DETAILED DESCRIPTION OF THE PREFERRED EMBODIMENTS

Before describing in detail an exemplary embodiment of a hybrid stent with polymeric and metallic components in accordance with the present invention, it is instructive to briefly describe a typical stent implantation procedure and the vascular conditions which are typically treated with stents. Referring now to FIG. 1, a stent 10 of the present invention is shown mounted on a catheter 11 having an inflation member 14 and slidable over guide wire 19. The stent and catheter are shown inside the lumen of an arterial vessel 16. The stent is shown positioned across a small amount of arterial plaque 15 adhering to the lumen of the artery. In some procedures, a stent is directly implanted without a prior procedure, such as balloon angioplasties. In other procedures, the plaque is the remainder of an arterial lesion which has been previously dilated or radially compressed against the walls of the artery, or has been partially removed from the artery. Lesion dilation is typically accomplished by an angioplasty procedure, while lesion removal is typically accomplished by an atherectomy procedure. These and other procedures for the treatment of arterial lesions are well known to those skilled in the art.

With most lesion treatment procedures, the treated artery suffers a degree of trauma, and in a certain percentage of cases may abruptly collapse or may slowly narrow over a period of time due to neointimal hyperplasia which is referred to as restenosis. To prevent either of these conditions, the treated artery is often fitted with a prosthetic device, such as the stent 10 of the present invention. The stent provides radial support for the treated vessel and thereby prevents collapse of the vessel 16, and further provides scaffolding to prevent plaque prolapse within the lumen. The stent may also be used to repair an arterial dissection, or an intimal flap, both of which are sometimes found in the coronary arteries, peripheral arteries and other vessels. In order to perform its function, the stent must be accurately placed across the lesion site. Therefore, it is critical that the stent be sufficiently radiopaque so that the physician can visually locate the stent under fluoroscopy during the implantation procedure. However, it is equally important that the stent not be too radiopaque. If the stent is overly radiopaque, i.e., too bright, the physician's view of the lumen is compromised. This makes assessment of subsequent restenosis difficult. In cases where the balloon markers are very close to the stent, the stent can blend in with the markers. Without precise visualization of the stent ends, accurate placement of the stent in a lesion, particularly in the case of an ostial lesion, can be compromised.

With continued reference to FIG. 1, in a typical stent placement procedure, a guiding catheter (not shown) is percutaneously introduced into the cardiovascular system of a patient through the femoral arteries by means of a conventional Seldinger technique, and advanced within a patient's vascular system until the distal end of the guiding catheter is positioned at a point proximal to the lesion site. A guide wire and the stent-delivery catheter 11 of the rapid exchange type are introduced through the guiding catheter with the guide wire sliding within the stent-delivery catheter. The guide wire is first advanced out of the guiding catheter into the arterial vessel 16 and is advanced across the arterial lesion. Prior to implanting the stent, the cardiologist may wish to perform an angioplasty or other procedure (e.g., atherectomy) in order to open and remodel the vessel and the diseased area.

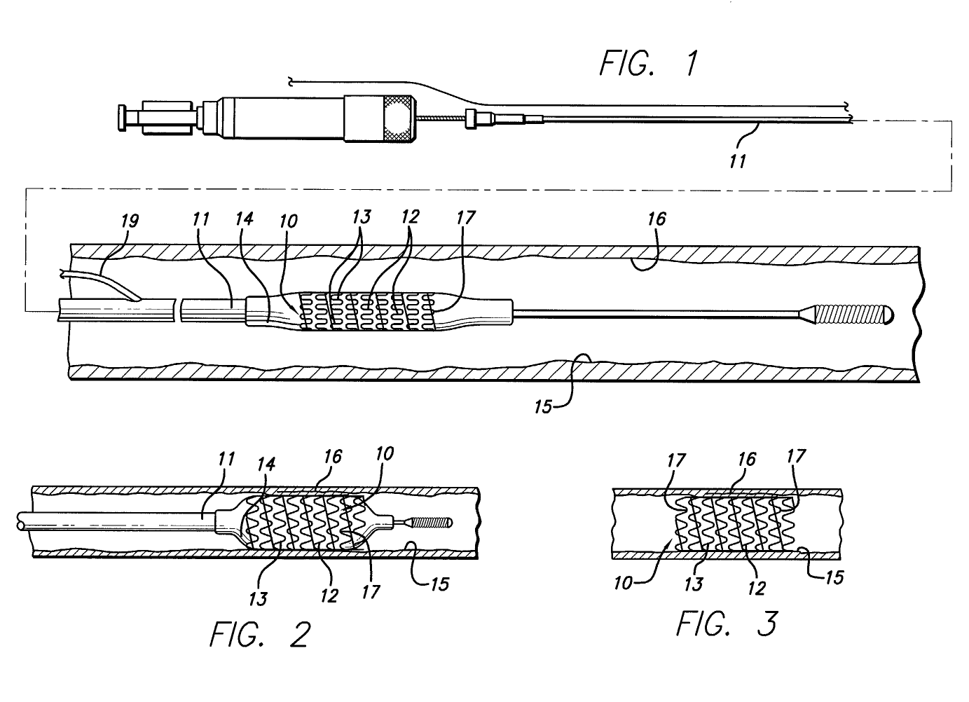

Referring to FIG. 2, the stent delivery catheter assembly 11 is advanced over the guide wire so that the stent 10 is positioned in the target area. The stent-delivery catheter is subsequently advanced over the previously positioned guide wire until the stent is properly positioned across the lesion.

Referring now to FIGS. 2 and 3, once in position, the dilation balloon 14 is inflated to a predetermined size to radially expand the stent 10 against the inside of the artery wall and thereby implant the stent within the lumen of the artery 16. The balloon 14 is then deflated to a small profile so that the stent-delivery catheter may be withdrawn from the patient's vasculature and blood flow resumed through the artery.

The metallic cylindrical rings 17 of this embodiment are formed from tubular members and relatively flat in transverse cross-section. Thus, after implantation into the artery 16 as shown in FIG. 3, minimal interference with blood flow occurs. Eventually the stent becomes covered with endothelial cell growth, which further minimizes blood flow interference. As should be appreciated by those skilled in the art that, while the above-described procedure is typical, it is not the only method used in placing stents.

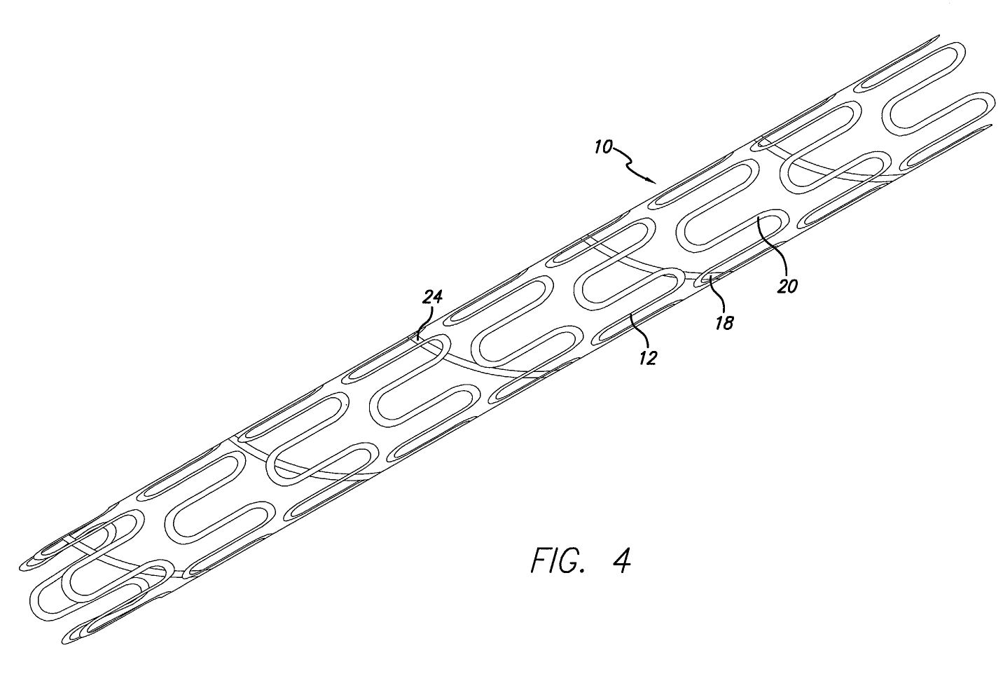

For the purposes of description it is beneficial to refer to this exemplary embodiment of the stent as being composed of metallic cylindrical rings 17 and a polymeric coil 18. The rings have a first, crimped delivery diameter and a second, larger implanted diameter. They are each expandable in the radial direction and aligned together along the longitudinal axis of the stent. The polymeric coil 18 similarly has a first crimped delivery diameter and a second expanded implanted diameter. The coil 18 and rings 12 are attached together and aligned longitudinally to form the stent. The rings can be attached on either the inner or outer surface of the coil. FIG. 4 shows the cylindrical rings 12 and polymeric coil 18 attached togther in the first delivery diameter. In this particular embodiment, the cylindrical rings 12 are attached on the outer surface of the polymeric coil 18.

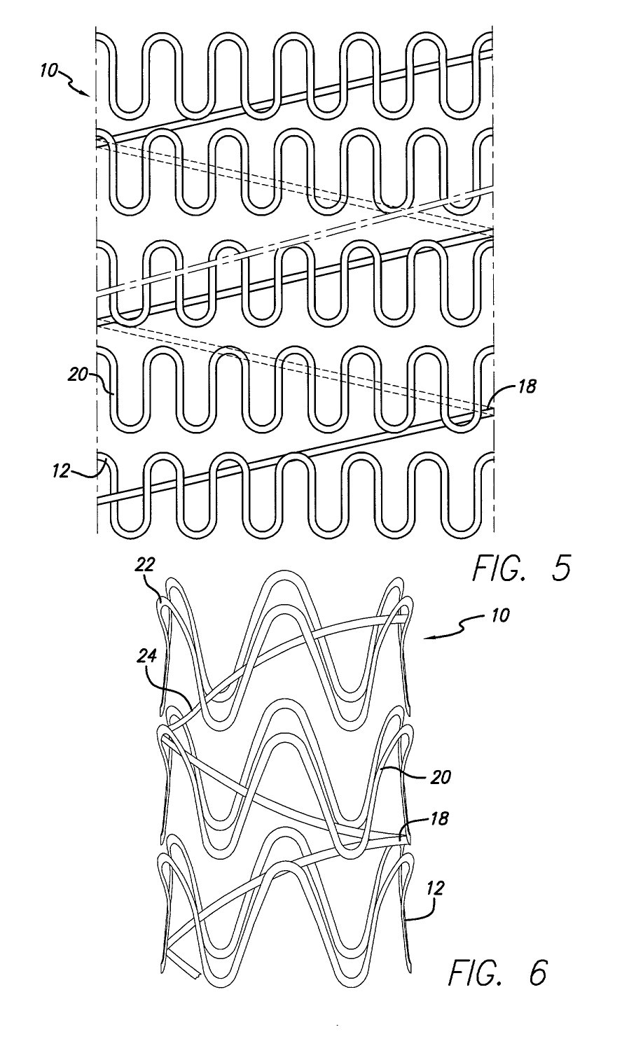

The properties of the stent 10 may vary by alteration of the cylindrical rings 12. FIG. 5 illustrates a plain view of a flattened section of the stent in its crimped state. The cylindrical rings have an undulating shape including peaks and valleys formed as U-shaped members 20 which are out of phase with adjacent cylindrical rings. The particular pattern and how many undulations, or the amplitude of the undulations, are chosen to fill particular mechanical requirements for the stent, such as radial stiffness. The number of cylindrical rings 12 incorporated into the stent can also vary according to design requirements such as radial stiffness and longitudinal flexibility.

With reference to FIG. 6, the cylindrical rings 12 are in the form of undulating portions. The undulating portion is made up of a plurality of U-shaped members 20 having a radius that more evenly distributes expansion forces over the various members. After the cylindrical rings 12 have been radially expanded, outwardly projecting edges 22 may be formed. That is, during radial expansion some of the U-shaped members may tip radially outwardly thereby forming outwardly projecting edges. These outwardly projecting edges can provide for a roughened outer wall surface of the stent 10 and assist in implanting the stent in the vascular wall by embedding into the vascular wall. In other words, outwardly projecting edges 22 embed into the vascular wall, for example arterial vessel 16, as depicted in FIG. 3. Depending upon the dimensions of stent 10 and the thickness of the various members making up the serpentine pattern, any of the U-shaped members 20 can tip radially outwardly to form a projecting edge 22.

Cylindrical rings 12 can be nested such that adjacent rings slightly overlap in the longitudinal direction so that one ring is slightly nested within the next ring and so on. The degree of nesting can be dictated primarily by the length of each cylindrical ring 12 the number of undulations in the rings, the thickness of the rings, and the radius of curvature, all in conjunction with the crimped or delivery diameter of the stent. If the rings are substantially nested one within the other, it may be difficult to crimp the stent to an appropriate delivery diameter without the various struts overlapping. It is also contemplated that the rings may be slightly nested even after the stent is expanded, which enhances vessel wall coverage. In some circumstances, it may not be desirable to nest one ring within the other, which is also contemplated by the invention.

The stent patterns shown in FIGS. 1-6 are for illustration purposes only and can vary in shape and size to accommodate different vessels or body lumens. Thus, rings connected by the coil can have any structural shapes and are not limited to the aforedescribed undulating rings with U-shaped members. For example, a variety of configurations incorporating generally Y-, W-, and Z-shaped members along with sine waves, loops, and sharp angles can be utilized according to design requirements. The cylindrical rings can also be formed with shape memory alloys, and radiopacity enhanced.

In keeping with the invention, the polymeric coil 18 is formed from a flexible polymeric material, that is bendable and flexible to enhance longitudinal and flexural flexibility of the stent 10. The polymeric coil can be formed with a spiraling pattern 24 to enable the stent to have higher flexibility and deliveribility than traditional all metal stents. The coil can also be formed in a plethora of different patterns according to design requirements. For example, the coil can be formed with more or less surface area, a greater or lower number of turns around the cylindrical rings, and a variety of other shapes according to design requirements.

The polymeric coil 18 when used in connection with the metallic cylindrical rings 12 enables the stent 10 to have higher flexibility and deliverability than all metal stents. Referring to FIG. 4, the cylindrical rings 12 are formed out of a metal, such as stainless steel and can be attached with a bonding agent to the outer surface of the polymeric coil 18. In this embodiment, the cylindrical rings 12 overlap the polymeric coil 18. The amount of overlap can vary according to design requirements. For example, it is possible for less than 5% of the metal constituting the cylindrical rings 12 to overlap with less than 30% the polymer constituting the polymeric coil. By comparison, an all metal stent generally consists of a series of metallic cylindrical rings interconnected by metallic links or struts. In the case of metallic stents where the rings and links are laser cut from a unitary thin-walled tube, the design of the stent is a compromise between flexibility and rigidity. The stent must be flexible enough to conform to the curvature of the body lumen it is inserted into and the stent must be rigid enough to remain in its expanded state once implanted.

In the stent of the present invention, the polymeric coil 18 is configured to enable the stent 10 to exceed the longitudinal flexibility of conventional metallic stents. Such flexibility is achieved by factors such as choice of polymeric material, type of spiraling pattern, and desired final size. To account for the required radial strength, the cylindrical rings 12 are formed from a metallic material as in conventional metallic stents. Because of the relatively small longitudinal length of the metallic rings and because the rings are not connected with metallic links, the flexibility provided by the polymeric coil is not significantly inhibited. Accordingly, it is possible with the present invention to produce a stent having radial strength equivalent to a conventional metallic stent while offering longitudinal flexibility exceeding that of the metallic stent. With the addition of more rings to the stent 10, radial stiffness can also be increased over a conventional stent while maintaining a high degree of flexibility.

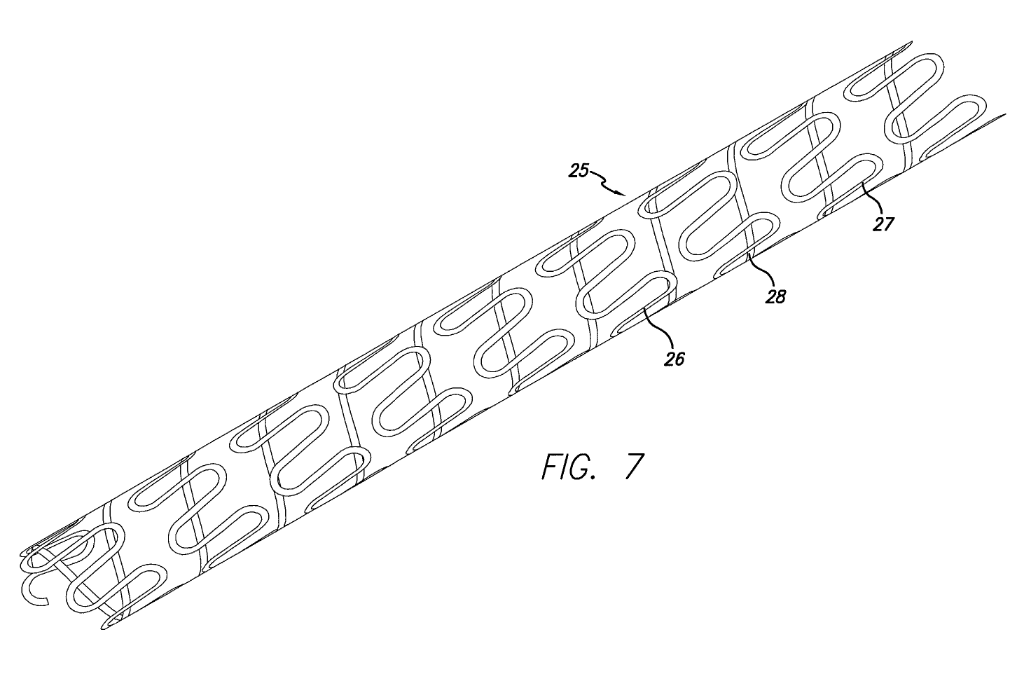

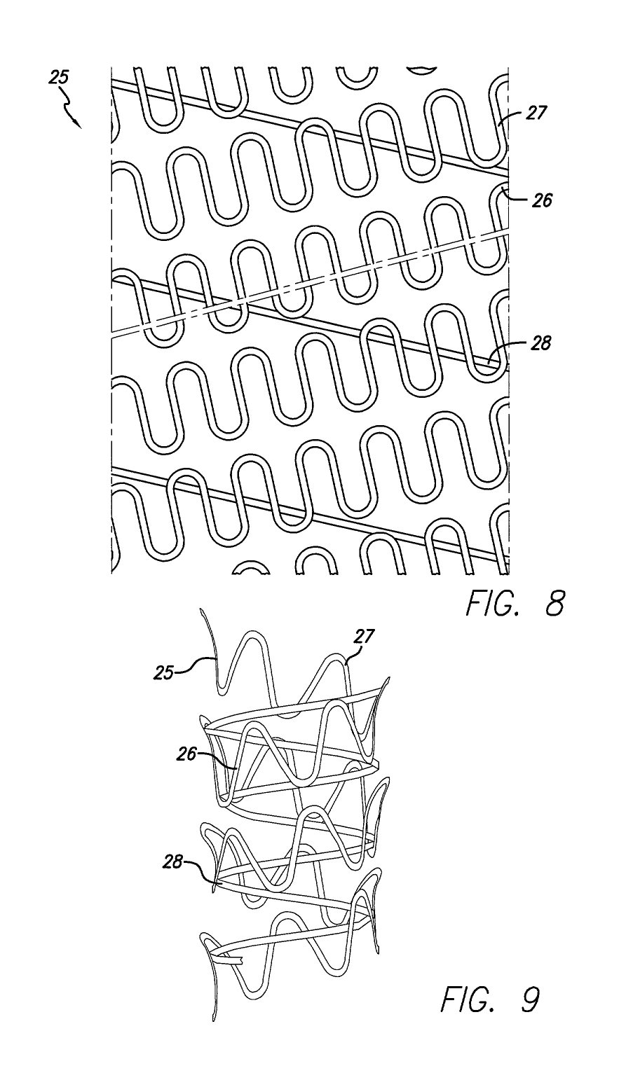

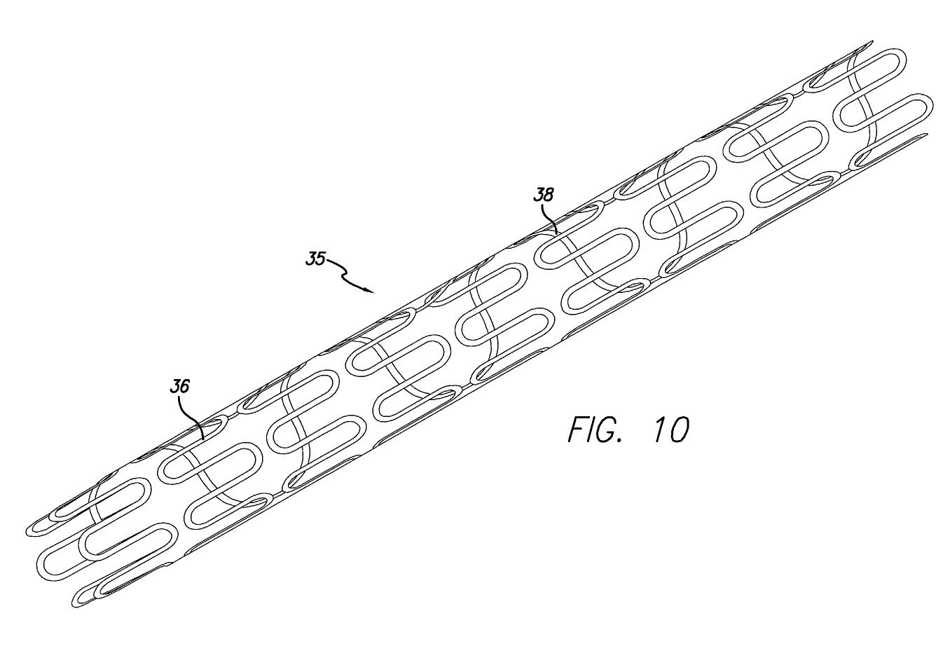

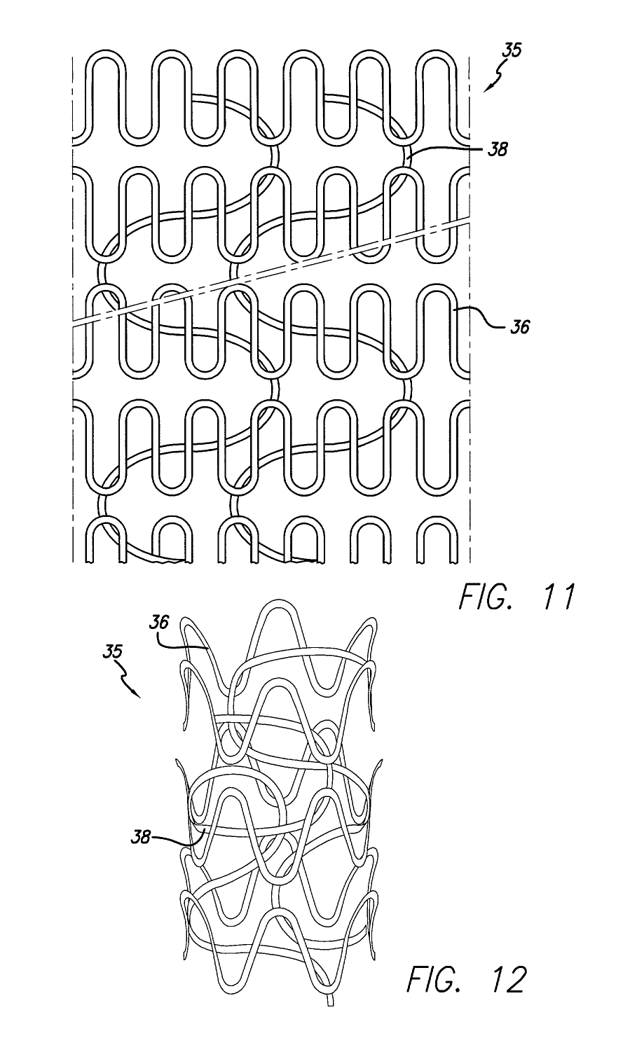

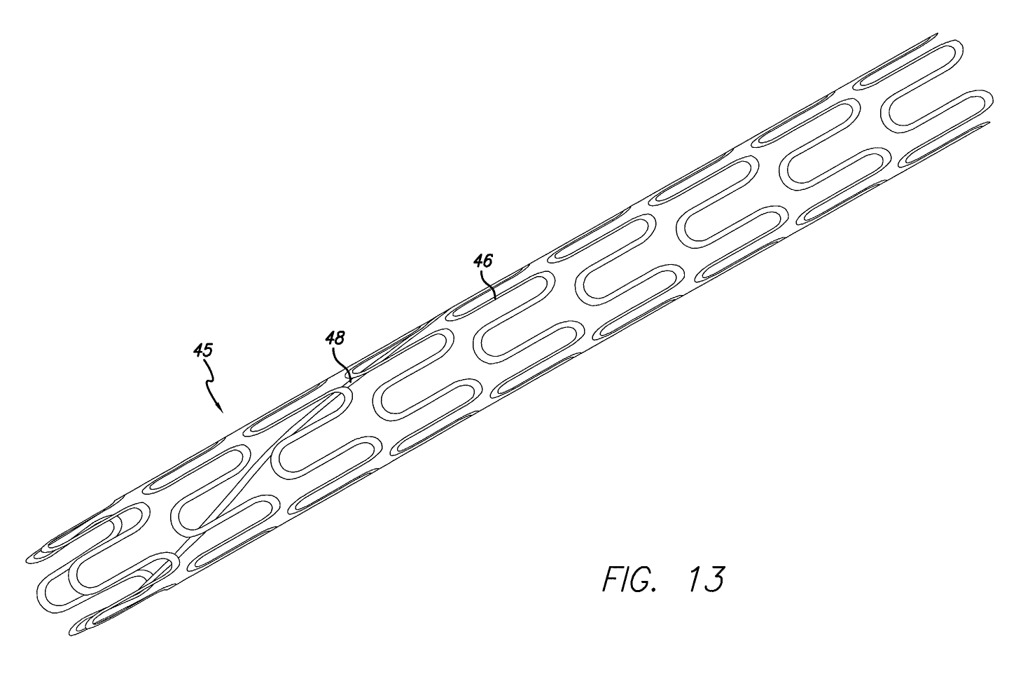

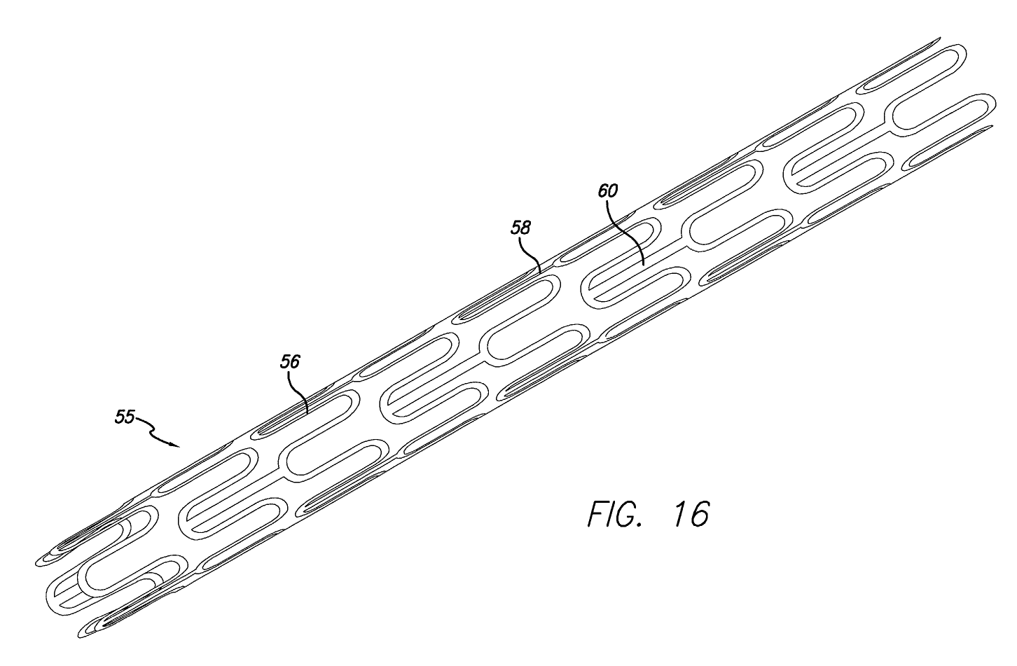

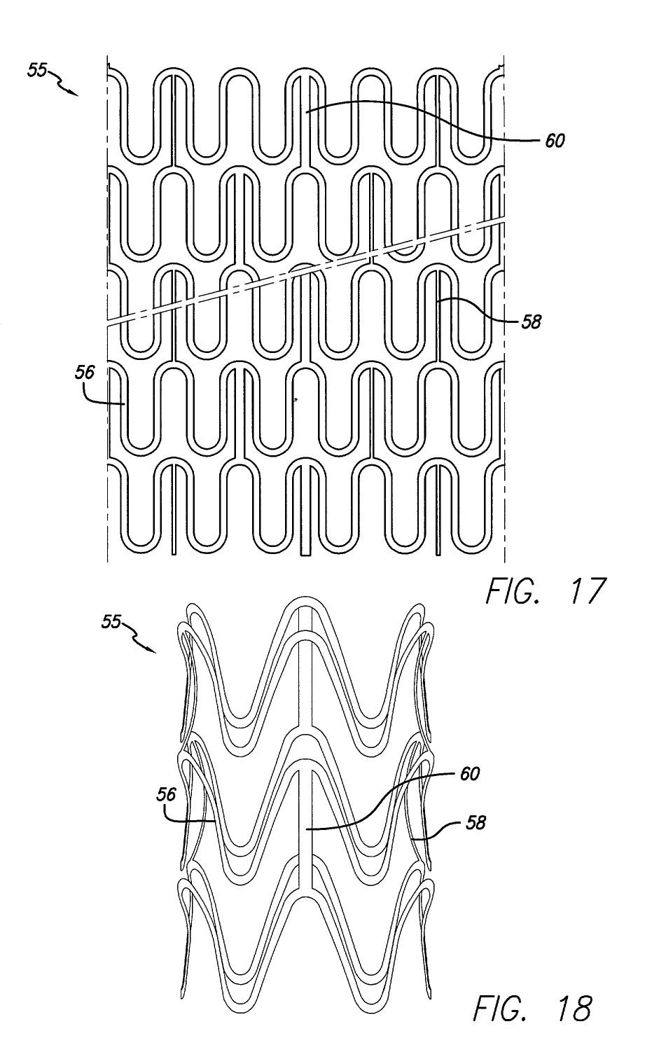

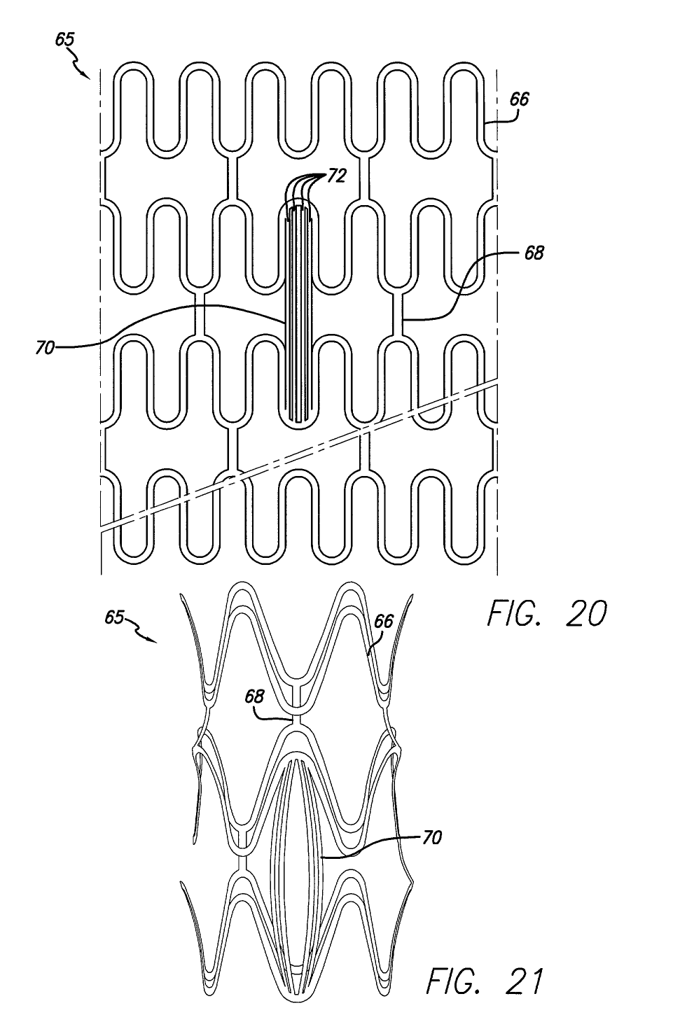

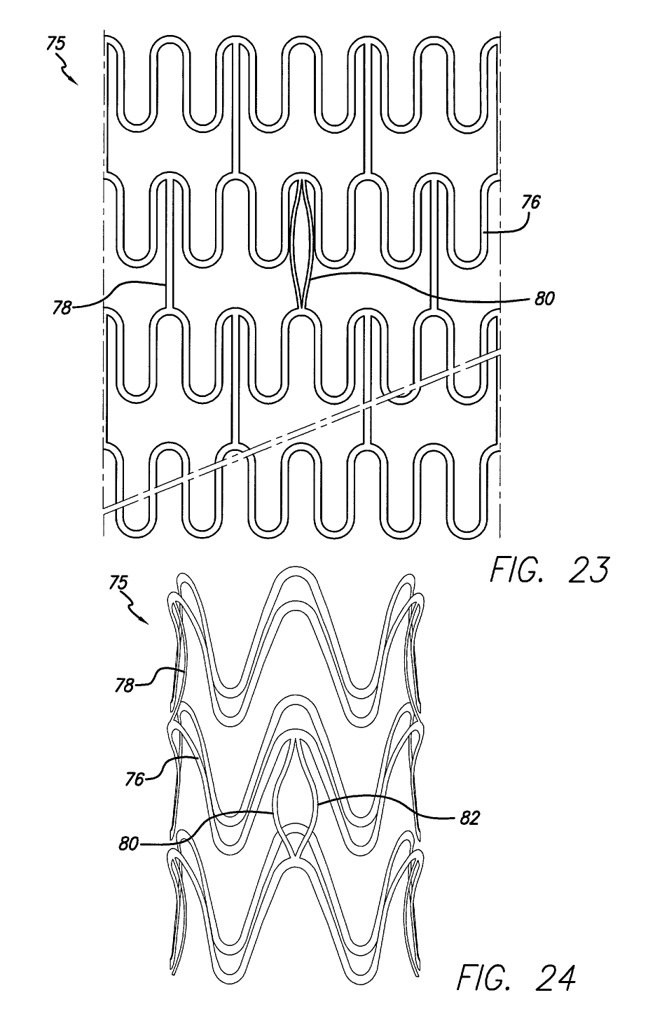

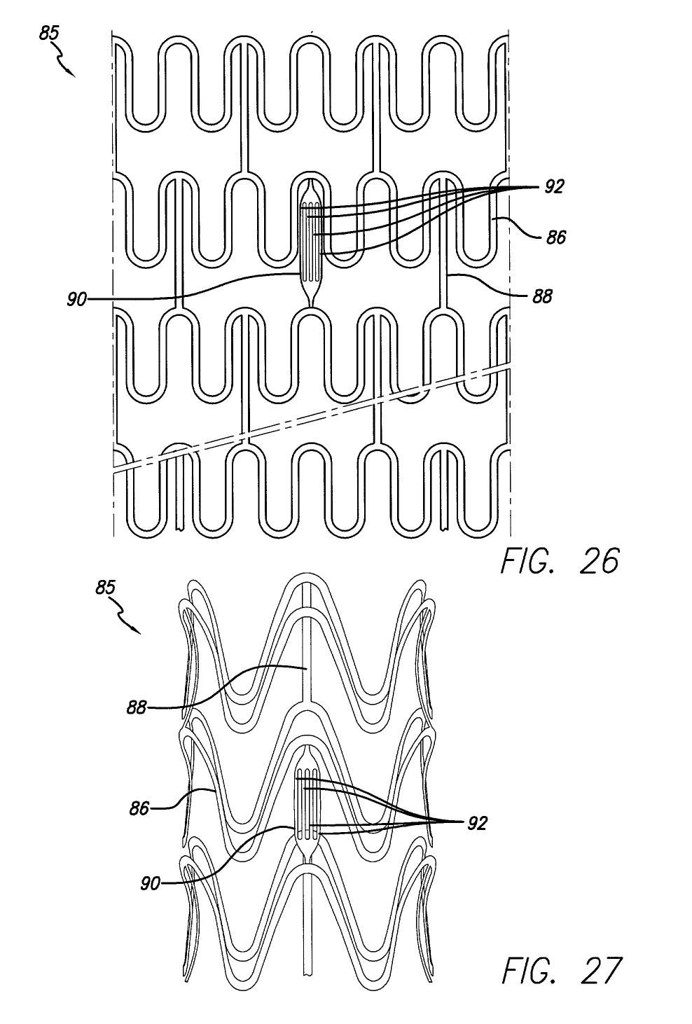

The multitude of stent embodiments illustrated in FIGS. 7 through 30 are similar to the above embodiment illustrated in FIGS. 1-6 in that each embodiment has essentially polymeric and metallic components including polymeric coils, wires, or links, and metallic rings or coils.

With reference to FIGS. 7-9, the cylindrical rings 26 are similar to the rings 12 illustrated in FIGS. 1-6 described above. The stent 25 is formed essentially in a coil-like manner where all rings 26 are continuously linked together to form a spiral spanning the length of the stent. Accordingly, the rings can be referred to also as a single metallic coil 27.

The polymeric coil 28 is attached to the inner surface of the metallic coil 27 and helps retain the balloon-expandable catheter when the stent is delivered. The retention can be enhanced with a polymeric material having a gripping surface. The polymeric coil 28 can also be attached to the outer surface of the metallic coil 27. The polymeric coil is also arranged in an opposing manner. More specifically, the value of the helix angle of the polymeric coil 28 is similar to the negative value of the helix angle of the metallic coil 27. Helix values of the coil and the rings are independent of each other and can vary according to design requirements. Further, the coils can be longitudinally offset from each other, therefore varying the locations of attachment between the two.

With reference to FIGS. 10-12, this embodiment of the stent 35 of the present invention includes cylindrical rings 36 longitudinally aligned along the stent's longitudinal axis with a series of wires 38 also aligned along the longitudinal axis. Similar to other embodiments of this invention, the wires 38 can be attached to either the inner or outer surfaces of the metallic cylindrical rings 36. As mentioned above, attaching the polymeric coil to the inside surface of the metallic rings can help in retaining the stent on the balloon catheter. The number of wires 38 can vary according to design requirements. Generally, as illustrated, the wires have an oscillating pattern that increases contact area with the cylindrical rings 36 when compared with a straight wire design. Formed from a polymer, the wires are also more flexible than a comparably dimensional series of metallic wires.

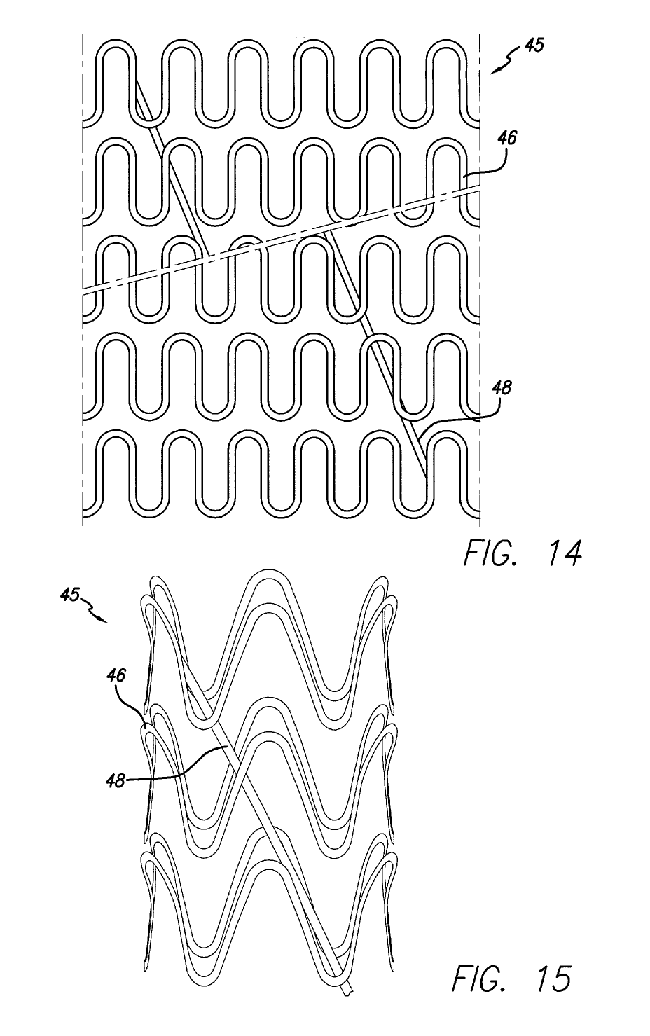

With reference to FIGS. 13-15, this embodiment of the stent 45 of the present invention includes cylindrical rings 46 longitudinally aligned along the stent's longitudinal axis. Similar to other embodiments, the polymeric wire 48 can be attached to either the inner or outer surface of the metallic cylindrical rings 46. In this embodiment a single wire is utilized for flexibility. When compared to an oscillating wire, the straight polymeric wire 48 may allow the stent to be more flexible due to the small amount of wire used to link the cylindrical rings together. The polymeric material also provides increased flexibility over a comparably dimensioned metallic wire. Radial strength is also sufficient because of the standard configuration of the rings 46. As in other embodiments, the cylindrical rings 46 are capable of compressing when crimped onto a balloon catheter and expanded to a semi-permanent state to hold a vessel open when delivered.

With reference to FIGS. 16-18, this embodiment of the stent 55 of the present invention includes cylindrical rings 56 aligned along the stent's longitudinal axis. Polymeric links 58,60 of varying width connect adjacent cylindrical rings 56. The links 58,60 have different widths and radial thickness varying from 0.001 to 0.01 inch. According to design requirements, the polymeric links can also incorporate polymers having durometers ranging from 1A to 99A and with frictional coefficients ranging from 0.1 to 0.9. This hybrid stent is particularly well suited to neurovascular applications. Flexibility is extremely important to negotiate through the carotid siphon and into the middle cerebral artery. The tremendous flexibility of the metal ring and polymer link greatly enhances flexibility.

A low durometer link will be relatively tacky and the resultant friction between the polymeric link and the balloon would help keep the stent crimped on the balloon during delivery. The mechanical compression of the low durometer polymer during crimping will also give a higher gripping force on the balloon than a comparably sized metallic link.

In a preferred embodiment, the links 58,60 are two different widths as shown in FIGS. 16-18. The narrow link 58 is a high durometer and low friction polymer. The wider link 60 is a low durometer polymer with a high friction polymer. As mentioned above, the types and sizes of polymers are chosen according to design requirements including stent retention and longitudinal flexibility.

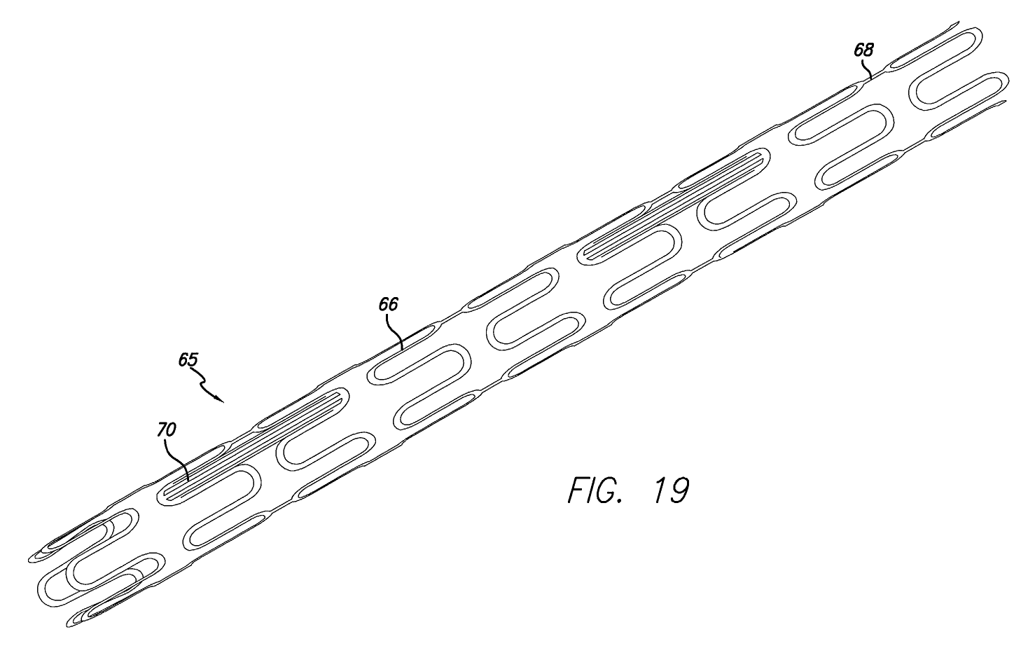

With reference to FIGS. 19-21, this embodiment of the stent 65 of the present invention includes cylindrical rings 66 longitudinally aligned along with polymeric links 68,70 connecting adjacent rings 66. This particular embodiment is well suited for wide neck aneurysms. Because the links 68,70 are polymeric and therefore easily formed into different shapes and made flexible, they can be configured specifically for this application. The polymer link 70 incorporates a four sublink 72 design while still maintaining the flexibility needed to reach the anterior cerebral, middle cerebral, or basilar artery. In this embodiment, the links 68,70 can be made wide or narrow with closely spaced connectors in the region of the aneurysm. The rings 66 as illustrated can be spaced farther apart than for a typical coronary application. The spacing helps to prevent occlusion of perforator vessels. Further, as illustrated in FIG. 19, adjacent rings 66 are placed so that adjacent peaks and valleys of adjacent rings are axially aligned.

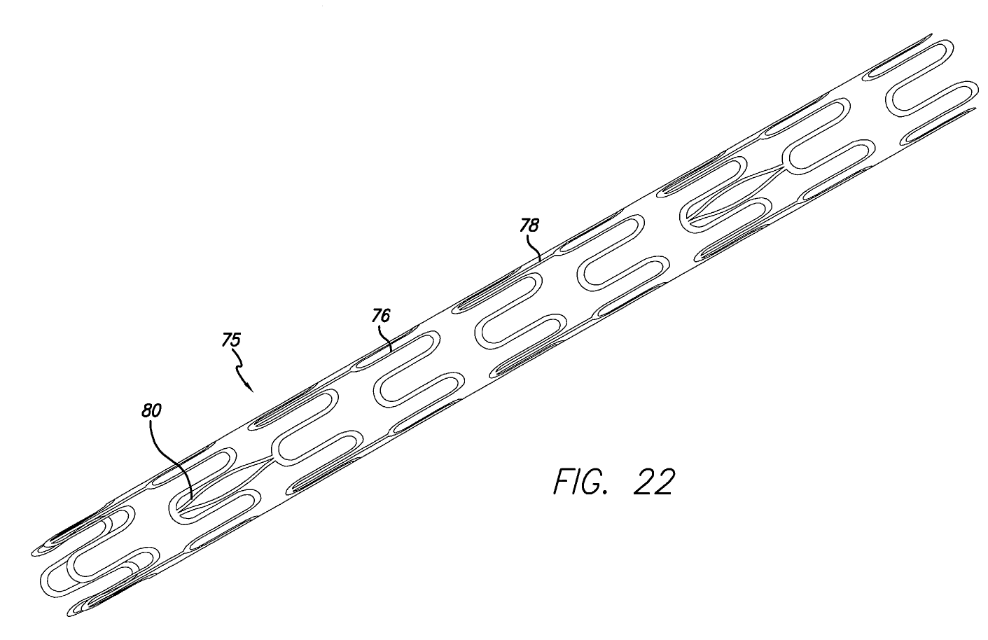

With reference to FIGS. 22-24, this embodiment of the stent 75 of the present invention includes cylindrical rings 76 longitudinally aligned and polymeric links 78,80 connecting the rings. Link 80 is formed in an oval-like shape with essentially two sublinks 82. This configuration provides flexibility while maintaining a wider area of contact than a more conventional straight one-piece link.

This embodiment is well suited for wide neck aneurysms. Like the stent 65 shown in FIGS. 19-21, this particular embodiment incorporates polymeric links 78,80 with the flexibility and configurations that enable the stent 75 to reach the anterior cerebral, middle cerebral, and basilar arteries. This embodiment also incorporates rings spaced farther apart than conventional coronary stents in order to prevent occlusion of perforator vessels.

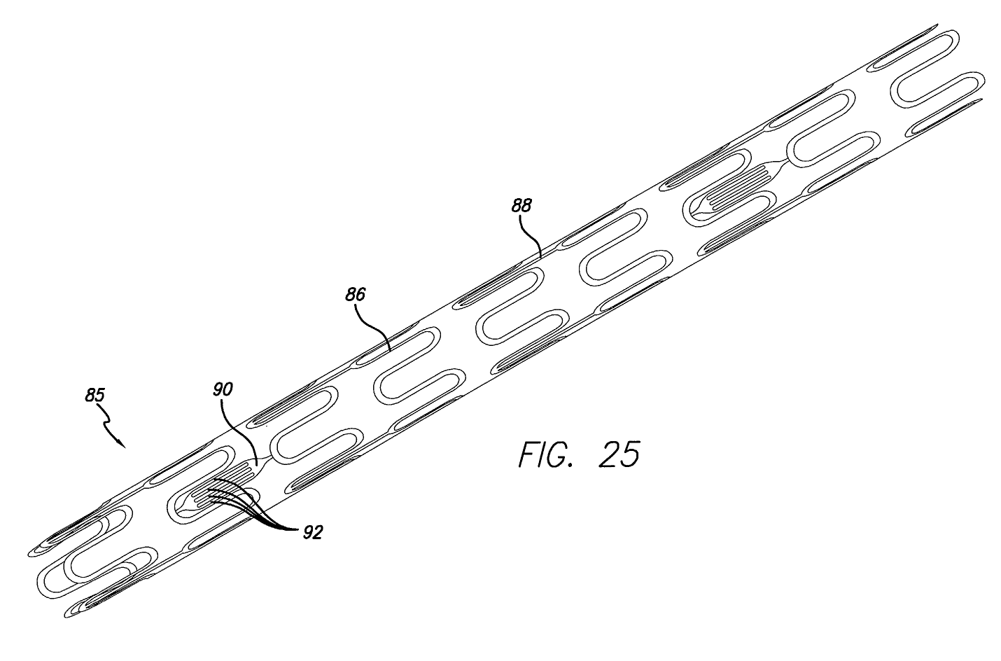

With reference to FIGS. 25-27, this embodiment of the stent 85 of the present invention includes cylindrical rings 86 longitudinally aligned and polymeric links 88,90 connecting the rings.

This embodiment is also well suited for wide neck aneurysms. Like stents 65,75 shown in FIGS. 19-21 and FIGS. 22-24, respectively, this embodiment incorporates polymeric links 88,90 flexible enough to enable the stent to reach the anterior cerebral, middle cerebral, and basilar arteries. Link 90 incorporates four sublinks 92. The sublinks 92 enable the link 90 to have similar flexible properties to a smaller link, yet cover a surface area similar to a large link. This enhanced flexibility and surface area coverage is well suited to neurovascular applications.

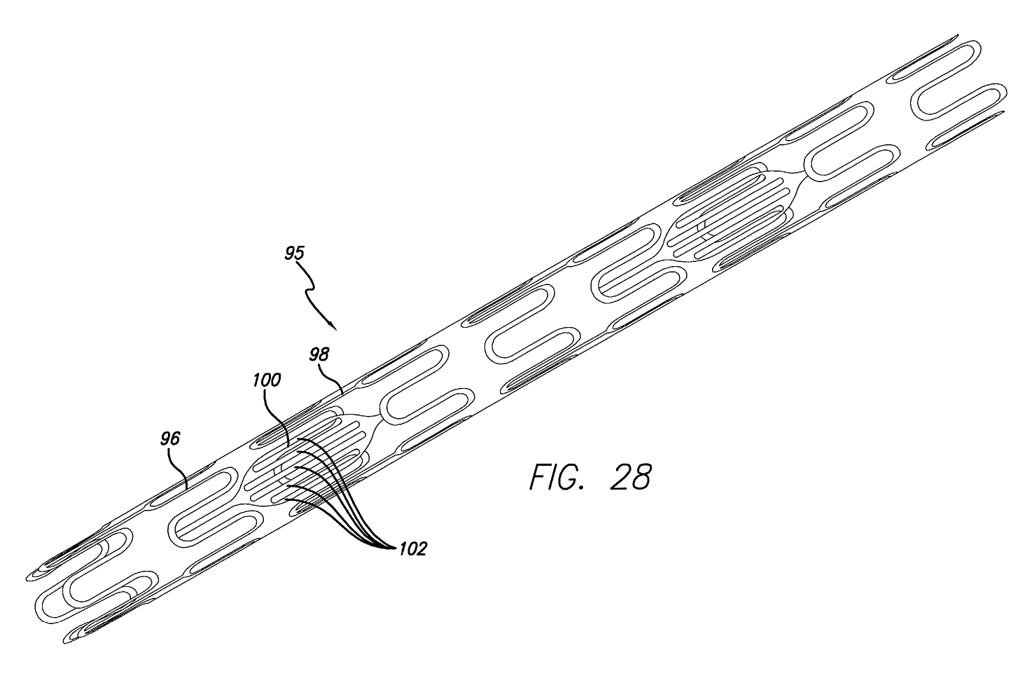

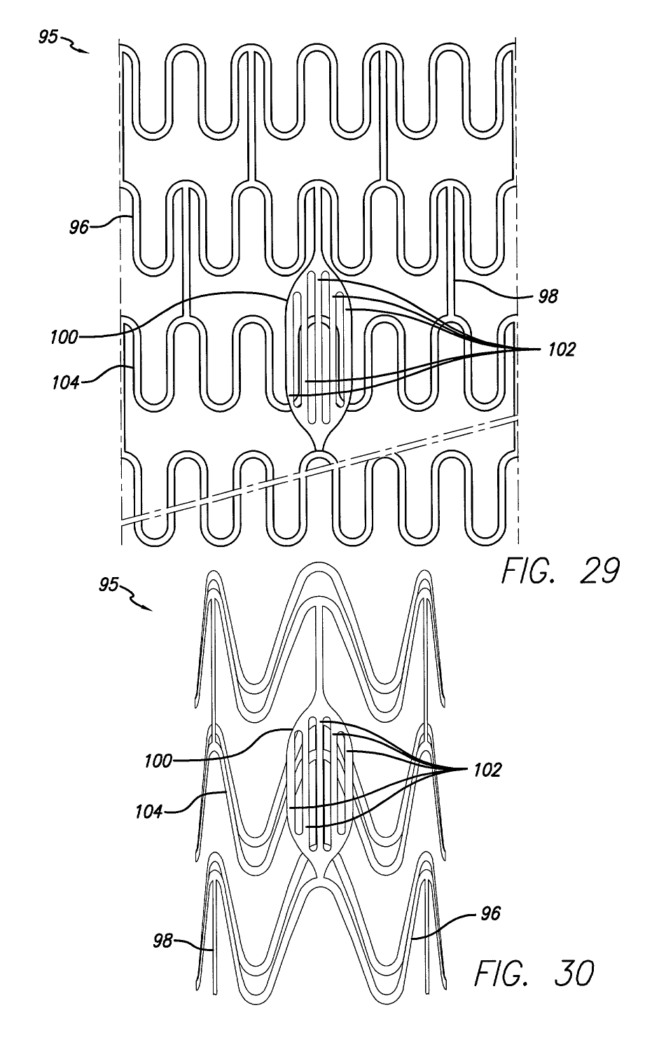

With reference to FIGS. 28-30, this embodiment of the stent 95 of the present invention includes cylindrical rings 96 longitudinally aligned and polymeric links 98,100 connecting the rings. This embodiment is also well suited for wide neck aneurysms. Like stents 65,75,85 shown in FIGS. 19-21, FIGS. 22-24, and FIGS. 25-27, respectively, this embodiment incorporates polymeric links 98,100 flexible enough to reach the anterior cerebral, middle cerebral, and basilar arteries. Link 100 incorporates five sublinks 102. The sublinks enable the link to be flexible like a smaller link, yet cover a large surface area similar to a large link. As depicted in FIG. 29, the link 100 spans three rings. In the illustration, there is no particular form of attachment to center ring 104 other than surface friction between the link 100 and the center ring 104. In accordance with design requirements including flexibility and rigidness, the link 100 may be bonded to the ring 104.

One method of making stents 10,25,35,45,55,65,75,85,95 of the invention is to form the polymeric components by conventional techniques, namely injection molding. In this aspect of the invention, the polymeric components can be first formed as a cylinder and then laser cut into a coil, series of wires, or series of links.

The polymeric material forming the polymeric component can be taken from the group of polymers consisting of polyurethanes, polyolefins, polyesters, polyamides, flouropolymers and their co-polymers, polyetherurethanes, polyesterurethanes, silicone, thermoplastic elastomer (C-flex), polyether-amide thermoplastic elastomer (Pebax), fluoroelastomers, fluorosilicone elastomer, styrene-butadiene rubber, butadiene-styrene rubber, polyisoprene, neoprene (polychloroprene), ethylene-propylene elastomer, chlorosulfonated polyethylene elastomer, butyl rubber, polysulfide elastomer, polyacrylate elastomer, nitrile, rubber, a family of elastomers composed of styrene, ethylene, propylene, aliphatic polycarbonate polyurethane, polymers augmented with antioxidents, polymers augmented with image enhancing materials, polymers having a proton (H+) core, polymers augmented with protons (H+), butadiene and isoprene (Kraton) and polyester thermoplastic elastomer (Hytrel).

The polymeric component can be relatively translucent or radiopaquely enhanced by the addition of suitably radiopaque materials. A material can also be compounded into the polymeric material to generate a magnetic susceptability artifact of the stent.

The metallic cylindrical rings or metallic coil can be fabricated from a highly radiopaque material such as tantalum, platinum, platinum iridium or a composite metal alloy such as a stainless steel with a platinum core to enhance the stent's resultant radiopacity. In another form, the rings or coil can be fabricated from a relatively translucent and suitably biocompatible material such as stainless steel.

One method of making the metallic component is to laser cut a thin-walled tubular member, such as stainless steel tubing to remove portions of the tubing in the desired pattern for the configuration, leaving relatively untouched the portions of the metallic tubing which are to form the rings or coil. In accordance with the invention, it is preferred to cut the tubing in the desired pattern by means of a machine-controlled laser as is well known in the art.

In one embodiment, a stainless steel tube may be Alloy type: 316L SS, Special Chemistry per ASTM F138-92 or ASTM F39-92 grade 2. Special Chemistry of type 316L per ASTM F138-92 or ASTM F139-92 Stainless Steel for Surgical Implants in weight percent.

| | |
|---|---|
| Carbon (C) | 0.03% max. |
| Manganese (Mn) | 2.00% max. |
| Phosphorous (P) | 0.025% max. |
| Sulphur (S) | 0.010% max. |
| Silicon (Si) | 0.75% max. |
| Chromium (Cr) | 17.00-19.00% |
| Nickel (Ni) | 13.00-15.50% |
| Molybdenum (Mo) | 2.00-3.00% |
| Nitrogen (N) | 0.10% max. |
| Copper (Cu) | 0.50% max. |
| Iron (Fe) | Balance |

The stent diameter is very small, so the tubing from which it is made must necessarily also have a small diameter. Typically the stent and rings have an outer diameter on the order of about 0.06 inch in the unexpanded condition, the same outer diameter of the tubing from which it is made, and can be expanded to an outer diameter of 0.1 inch or more. The wall thickness of the tubing is about 0.003 inch.

The tubing is mounted in a rotatable collet fixture of a machine-controlled apparatus for positioning the tubing relative to a laser. According to machine-encoded instructions, the tubing is rotated and moved longitudinally relative to the laser which is also machine controlled. The laser selectively removes the material from the tubing by ablation and a pattern is cut into the tube. The tube is therefore cut into the discrete pattern of the finished cylindrical rings.

The process of cutting a pattern for the rings into the tubing is automated except for loading and unloading the length of tubing. In one example, a CNC-opposing collet fixture for axial rotation of the length of tubing is used in conjunction with a CNC X/Y table to move the length of tubing axially relatively to a machine-controlled laser. The entire space between collets can be patterned using the CO₂ laser set-up of the foregoing example. The program for control of the apparatus is dependent on the particular configuration used and the pattern to be ablated in the coating.

Cutting a fine structure (0.0035 inch web width) requires minimal heat input and the ability to manipulate the tube with precision. It is also necessary to support the tube yet not allow the stent structure to distort during the cutting operation. In order to successfully achieve the desired end results, the entire system must be configured very carefully. The tubes are typically made of stainless steel with an outside diameter of 0.060 inch to 0.066 inch and a wall thickness of 0.002 inch to 0.004 inch. These tubes are fixtured under a laser and positioned utilizing a CNC to generate a very intricate and precise pattern. Due to the thin wall and the small geometry of the ring or coil pattern (0.0035 inch typical web width), it is necessary to have very precise control of the laser, its power level, the focused spot size, and the precise positioning of the laser cutting path.

In order to minimize the heat input into the stent structure, which prevents thermal distortion, uncontrolled bum out of the metal, and metallurgical damage due to excessive heat, and thereby produce a smooth debris free cut, a Q-switched Nd-YAG, typically available from Quantronix of Hauppauge, N.Y., that is frequency doubled to produce a green beam at 532 nanometers is utilized. Q-switching produces very short pulses (<100 nS) of high peak powers (kilowatts), low energy per pulse (≦3 mJ), at high pulse rates (up to 40 kHz). The frequency doubling of the beam from 1.06 microns to 0.532 microns allows the beam to be focused to a spot size that is 2 times smaller, therefore increasing the power density by a factor of 4 times. With all of these parameters, it is possible to make smooth, narrow cuts in the stainless tubes in very fine geometries without damaging the narrow series of undulations having peaks and valleys that make up the stent structure. Hence, the system of the present invention makes it possible to adjust the laser parameters to cut narrow kerf width which will minimize the heat input into the material.

The positioning of the tubular structure requires the use of precision CNC equipment such as that manufactured and sold by Anorad Corporation. In addition, a unique rotary mechanism has been provided that allows the computer program to be written as if the pattern were being cut from a flat sheet. This allows both circular and linear interpolation to be utilized in programming. Since the finished structure of the rings is very small, a precision drive mechanism can be used that supports and drives both ends of the tubular structure as it is cut. Since both ends are driven, they must be aligned and precisely synchronized, otherwise the tubular structure would twist and distort as it is being cut. Depending on manufacturing convenience, the rings or coil can also be formed in this manner, from a radiopaque material. After the above operations, the rings can be separated and individually processed, or processed while still connected in tubular form and later separated.

The optical system which expands the original laser beam, delivers the beam through a viewing head and focuses the beam onto the surface of the tube, incorporates a coaxial gas jet and nozzle that helps to remove debris from the kerf and cools the region where the beam interacts with the material as the beam cuts and vaporizes the metal. It is also necessary to block the beam as it cuts through the top surface of the tube and prevent the beam, along with the molten metal and debris from the cut, from impinging on the opposite surface of the tube.

In addition to the laser and the CNC positioning equipment, the optical delivery system includes a beam expander to increase the laser beam diameter, a circular polarizer, typically in the form of a quarter wave plate, to eliminate polarization effects in metal cutting, provisions for a spatial filter, a binocular viewing head and focusing lens, and a coaxial gas jet that provides for the introduction of a gas stream that surrounds the focused beam and is directed along the beam axis. The coaxial gas jet nozzle (0.018 inch I.D.) is centered around the focused beam with approximately 0.010 inch between the tip of the nozzle and the tubing. The jet is pressurized with oxygen at 20 psi and is directed at the tube with the focused laser beam exiting the tip of the nozzle (0.018 inch dia.). The oxygen reacts with the metal to assist in the cutting process very similar to oxyacetylene cutting. The focused laser beam acts as an ignition source and controls the reaction of the oxygen with the metal. In this manner, it is possible to cut the material with a very fine kerf with precision. In order to prevent burning by the beam and/or molten slag on the far wall of the tube I.D., a stainless steel mandrel (approx. 0.034 inch dia.) is placed inside the tube and is allowed to roll on the bottom of the tube as the pattern is cut. This acts as a beam/debris block protecting the far wall I.D.

Alternatively, this may be accomplished by inserting a second tube inside the ring tubing which has an opening to trap the excess energy in the beam which is transmitted through the kerf along which collecting the debris that is ejected from the laser cut kerf. A vacuum or positive pressure can be placed in this shielding tube to remove the collection of debris.

Another technique that could be utilized to remove the debris from the kerf and cool the surrounding material would be to use the inner beam blocking tube as an internal gas jet. By sealing one end of the tube and making a small hole in the side and placing it directly under the focused laser beam, gas pressure could be applied creating a small jet that would force the debris out of the laser cut kerf from the inside out. This would eliminate any debris from forming or collecting on the inside of the stent structure. It would place all the debris on the outside. With the use of special protective coatings, the resultant debris can be easily removed.

In most cases, the gas utilized in the jets may be reactive or non-reactive (inert). In the case of reactive gas, oxygen or compressed air is used. Inert gas such as argon, helium, or nitrogen can be used to eliminate any oxidation of the cut material. The result is a cut edge with no oxidation, but there is usually a tail of molten material that collects along the exit side of the gas jet that must be mechanically or chemically removed after the cutting operation.

The cutting process utilizing oxygen with the finely focused green beam results in a very narrow kerf (approx. 0.0005 inch) with the molten slag re-solidifying along the cut. This traps the cut out scrap of the pattern requiring further processing. In order to remove the slag debris from the cut allowing the scrap to be removed from the remaining stent pattern, it is necessary to soak the cut tube in a solution of HCL for approximately 8 minutes at a temperature of approximately 55° C. Before it is soaked, the tube is placed in a bath of alcohol/water solution and ultrasonically cleaned for approximately 1 minute to remove the loose debris left from the cutting operation. After soaking, the tube is then ultrasonically cleaned in the heated HCL for 1-4 minutes depending upon the wall thickness. To prevent cracking/breaking of the struts attached to the material left at the two ends of the ring or coil pattern due to harmonic oscillations induced by the ultrasonic cleaner, a mandrel is placed down the center of the rings or coil during the cleaning/scrap removal process. At completion of this process, the rings or coil are rinsed in water. They are now ready for electropolishing.

The rings or coil are preferably electrochemically polished in an acidic aqueous solution such as a solution of ELECTRO-GLO#300, sold by ELECTRO-GLO Co., Inc. in Chicago, Ill., which is a mixture of sulfuric acid, carboxylic acids, phosphates, corrosion inhibitors and a biodegradable surface active agent. The bath temperature is maintained at about 110°-1350° F. and the current density is about 0.4 to about 1.5 amps per in.2. Cathode to anode area should be at least about two to one.

The stents may be further treated if desired, for example by applying a biocompatible coating.

It will be apparent that both focused laser spot size and depth of focus can be controlled by selecting beam diameter and focal length for the focusing lens. It will be apparent that increasing laser beam diameter, or reducing lens focal length, reduces spot size at the cost of depth of field.

Direct laser cutting produces edges which are essentially perpendicular to the axis of the laser cutting beam, in contrast with chemical etching and the like which produce pattern edges which are angled. Hence, the laser cutting process essentially provides stent cross-sections, from cut-to-cut, which are square or rectangular, rather than trapezoidal. The cross-sections have generally perpendicular edges formed by the laser cut. The resulting cylindrical rings or coil provide superior performance.

Other methods of forming the cylindrical rings or coil of the present invention can be used, such as chemical etching; electric discharge machining; laser cutting a flat sheet and rolling it into a cylinder; and the like, all of which are well known in the art at this time.

The stent of the present invention also can include a superelastic material. The term “superelastic” refers to an isothermal transformation, more specifically stress inducing a martensitic phase from an austenitic phase. Alloys having superelastic properties generally have at least two phases: a martensitic phase, which has a relatively low tensile strength and which is stable at relatively low temperatures, and an austenitic phase, which has a relatively high tensile strength and which is stable at temperatures higher than the martensitic phase. The austenitic phase also typically has better corrosion properties. Superelastic characteristics generally allow the metal stent to be deformed by collapsing and deforming the stent and creating stress which causes the metal to change to the martensitic phase. The stent is restrained in the deformed condition to facilitate the insertion into a patient's body, with such deformation causing the phase transformation. Once within the body lumen, the restraint on the stent is removed, thereby reducing the stress therein so that the superelastic stent can return towards original undeformed shape by the transformation back to the austenitic phase. A basic discussion of this phenomenon can be found in Wayman and Duerig, “An Introduction to Martensite and Shape Memory,” which appears in Engineering Aspects Of Shape Memory Alloys, Duerig et al. editors (Butterworth-Heinemann Ltd. 1990, Great Britain.) The metallic cylindrical rings or coil can be formed from a superelastic material such as NiTi and undergo an isothermal transformation when stressed. The stent is first compressed to a delivery diameter, thereby creating stress in the NiTi alloy so that the NiTi is in a martensitic state having relatively low tensile strength. While still in the martensitic phase, the stent is mounted onto a catheter by known methods.

When stress is applied to a specimen of a metal such as nitinol exhibiting superelastic characteristics at a temperature at or above that which the transformation of the martensitic phase to the austenitic phase is complete, the specimen deforms elastically until it reaches a particular stress level where the alloy then undergoes a stress-induced phase transformation from the austenitic phase to the martensitic phase. As the phase transformation progresses, the alloy undergoes significant increases in strain with little or no corresponding increases in stress. The strain increases while the stress remains essentially constant until the transformation of the austenitic phase to the martensitic phase is complete. Thereafter, further increase in stress is necessary to cause further deformation. The martensitic metal first yields elastically upon the application of additional stress and then plastically with permanent residual deformation.

If the load on the specimen is removed before any permanent deformation has occurred, the martensite specimen will elastically recover and transform back to the austenitic phase. The reduction in stress first causes a decrease in strain. As stress reduction reaches the level at which the martensitic phase transforms back into the austenitic phase, the stress level in the specimen will remain essentially constant (but less than the constant stress level at which the austenitic crystalline structure transforms to the martensitic crystalline structure until the transformation back to the austenitic phase is complete); i.e., there is significant recovery in strain with only negligible corresponding stress reduction. After the transformation back to austenite is complete, further stress reduction results in elastic strain reduction. This ability to incur significant strain at relatively constant stress upon the application of a load and to recover from the deformation upon the removal of the load is commonly referred to as superelasticity

To attach the cylindrical rings or coil to the polymeric coil, wire, or links, adhesive can be applied to the polymeric component and to corresponding points of intersection on the metallic rings or coil. The adhesive can be any biocompatible adhesive that is well known, such as a cyanoacrylite-based adhesive. Several adhesives can be used including Locitite 401, 1-06FL, and M-11FL, the latter two of which are urethane-based adhesives. Other adhesives can be used without departing from the spirit and scope of the invention. Other methods of connecting the polymeric and metallic components also can be used without departing from the scope of the invention. For example, the metallic rings or coil can incorporate slots for accepting the coils or wire. To secure the two surfaces, an interference fit can be utilized or an adhesive as mentioned above can be used. Other types of interference fits and adhesives also can be utilized according to design requirements.

The stent of the present invention may also be used in connection with a therapeutic agent to perform a variety of functions, from preventing blood clots to promoting healing. When compared with conventional all metal stents, the packed cell structure of the stent of the present invention enables the delivery of the drug to the arterial walls to be more uniform. The lack of uniformity of drug distribution to the arterial walls is one of the main drawbacks of the current metallic drug delivery stents.

As an example, an active agent loaded into or coated on the polymeric component can inhibit the activity of vascular smooth muscle cells. Similarly, an active agent coated on the metallic component can also inhibit the activity of vascular smooth muscle cells. More specifically, the active agent is aimed at inhibiting abnormal or inappropriate migration and proliferation of smooth muscle cells. The active agent can also include any substance capable of exerting a therapeutic or prophylactic effect in the practice of the present invention. The agent can also be for enhancing wound healing in a vascular site or improving the structural and elastic properties of the vascular site. The dosage or concentration of the active agent required to produce a favorable therapeutic effect should be less than the level at which the active agent produces toxic effects and greater than the level at which non-therapeutic results are obtained. The dosage or concentration of the active agent required to inhibit the desired cellular activity of the vascular region can depend upon factors such as the particular circumstances of the patient; the nature of the trauma; the nature of the therapy desired; the time over which the ingredient administered resides at the vascular site; and if other therapeutic agents are employed, the nature and type of the substance or combination of substances. Therapeutic effective dosages can be determined empirically, for example by infusing vessels from suitable animal model systems and using immunohistochemical, fluorescent or electron microscopy methods to detect the agent and its effects, or by conducting suitable in vitro studies. Standard pharmacological test procedures to determine dosages are understood by one of ordinary skill in the art.

Examples of therapeutic agents that are available include rapamycin, actinomycin D (ActD), or derivatives and analogs thereof ActD is manufactured by Sigma-Aldrich, 1001 West Saint Paul Avenue, Milwaukee Wis. 53233, or COSMEGEN, available from Merck. Synonyms of actinomycin D include dactinomycin, actinomycin IV, actinomycin l1, actinomycin X1, and actinomycin C1. Examples of agents include other antiproliferative substances as well as antineoplastic, antiinflammatory, antiplatelet, anticoagulant, antifibrin, antithomobin, antimitotic, antibiotic, and antioxidant substances. Examples of antineoplastics include taxol (paclitaxel and docetaxel). Examples of antiplatelets,, anticoagulants, antifibrins, and antithrombins include sodium heparin, low molecular weight heparin, hirudin, argatroban, forskolin, vapiprost, prostacyclin and prostacyclin analogs, dextran, D-phe-pro-arg-chloromethylketone (synthetic antithrombin), dipyridamole, glycoprotein, llb/llla platelet membrane receptor antagonist, recombinant hirudin, thrombin inhibitor (available from Biogen), and 7E-3B® (an antiplatelet drug from Centocore). Examples of antimitotic agents include methotrexate, azathioprine, vincristine, vinblastine, fluorouracil, adriamycin, and mutamycin. Examples of cytostatic or antiproliferative agents include angiopeptin (a somatostatin analog from Ibsen), angiotensin converting enzyme inhibitors such as Captopril (available from Squibb), Cilazapril (available from Hoffman-LaRoche), or Lisinopril (available from Merck); calcium channel blockers (such as Nifedipine), colchicine fibroblast growth factor (FGF) antagonists, fish oil (omega 3-fatty acid), histamine antagonist, Lovastatin (an inhibitor of HMG-CoA reductase, a cholesterol lowering drug from Merck), monoclonal antibodies (such as PDGF receptors), nitroprusside, phosphodiesterase inhibitors, prostaglandin inhibitor (available from Glazo), Seramin (a PDGF antagonist), serotonin blockers, steroids, thioprotease inhibitors, triazolopyrimidine (a PDGF antagonist), and nitric oxide. Other therapeutic substances or agents which may be appropriate include alpha-interferon, genetically engineered epithelial cells, and dexamethasone.

While the invention has been illustrated and described herein in terms of its use as an intravascular stent, it will be apparent to those skilled in the art that the stent can be used in other body lumens. Further, particular sizes and dimensions, number of undulations per ring, materials used, and the like have been described herein and are provided as examples only. Other modifications and improvements may be made without departing from the scope of the invention.

What is claimed:

1. An intravascular stent, comprising:
a plurality of metallic cylindrical rings with a first delivery diameter and a second implanted diameter, the rings being expandable in a radial direction and aligned along a longitudinal axis of the stent; and
a polymeric coil with a first delivery diameter and a second implanted diameter, the coil being expandable in a radial direction and having an outer surface and an inner surface and being aligned along a longitudinal axis of the stent.

2. The stent of claim 1, wherein the cylindrical rings are attached to the outer surface of the polymeric coil.

3. The stent of claim 1, wherein the cylindrical rings are attached to the inner surface of the polymer coil.

4. The stent of claim 1, wherein longitudinal resistance to bending is less than a metallic stent having the same size and shape.

5. The stent of claim 1, wherein radial resistance to compression is at least equal to a metallic stent having the same size and shape.

6. The stent of claim 1, wherein the cylindrical rings and the coil are continuously coupled together in both the first delivery diameter and second implanted diameter respectively.

7. The stent of claim 1, wherein a plurality of protrusions extend radially outward of the cylindrical rings in the second implanted diameter.

8. The stent of claim 1, wherein the rings are attached to the coil with a bonding agent.

9. The stent of claim 1, wherein the rings fit within slots in the outer surface of the coil.

10. The stent of claim 1, wherein the coil radially compresses when the stent is crimped onto a catheter and radially expands when the stent is deployed from the catheter.

11. The stent of claim 1, wherein the metallic material forming the cylindrical rings is taken from the group of metals consisting of stainless steel, titanium, tantalum, nickel titanium, cobalt-chromium, gold, paladium, platinum and iradium.

12. The stent of claim 1, wherein the polymeric material forming the coil is taken from the group of polymers consisting of polyurethanes, polyetherurethanes, polyesterurethanes, silicone, thermoplastic elastomer (C-flex), polyether-amide thermoplastic elastomer (Pebax), fluoroelastomers, fluorosilicone elastomer, styrene-butadiene rubber, butadiene-styrene rubber, polyisoprene, neoprene (polychloroprene), ethylene-propylene elastomer, chlorosulfonated polyethylene elastomer, butyl rubber, polysulfide elastomer, polyacrylate elastomer, nitrile, rubber, a family of elastomers composed of styrene, ethylene, propylene, aliphatic polycarbonate polyurethane, polymers augmented with antioxidents, polymers augmented with image enhancing materials, polymers having a proton (H+) core, polymers augmented with protons (H+), butadiene and isoprene (Kraton) and polyester thermoplastic elastomer (Hytrel).

13. The stent of claim 1, wherein the plurality of cylindrical rings have undulations comprising a plurality of peaks and valleys.

14. The stent of claim 13, wherein a plurality of cylindrical rings are bonded to the coil at points in between the peaks and valleys of the cylindrical rings.

15. The stent of claim 13, wherein the peaks and valleys of a plurality of cylindrical rings form U-shaped portions.

16. The stent of claim 13, wherein the peaks and valleys of a plurality of cylindrical rings form Y-shaped portions.

17. The stent of claim 13, wherein the peaks and valleys of a plurality of cylindrical rings form W-shaped portions.

18. The stent of claim 13, wherein the peaks of each cylindrical ring are axially aligned with the valleys of each adjacent cylindrical ring.

19. The stent of claim 13, wherein the peaks of each cylindrical ring are axially aligned with the peaks of each adjacent cylindrical ring.

20. The stent of claim 1, wherein the stent is self-expanding.

21. The stent of claim 20, wherein the material forming the cylindrical rings embodies shape memory characteristics.

22. The stent of claim 21, wherein the shape memory material is a superelastic material.

23. The stent of claim 22, wherein the superelastic material is nickel titanium.

24. The stent of claim 1, wherein the coil is loaded with a therapeutic drug.

25. The stent of claim 1, wherein the coil is coated with a bioactive coating.

26. The stent of claim 1, wherein the stent is bio-compatible.

27. The stent of claim 1, wherein the stent is non-biodegradable.

28. The stent of claim 1, wherein a material is compounded into the polymeric coil to generate a magnetic susceptibility artifact of the stent.

29. The stent of claim 1, wherein the polymeric coil includes a material therein to enhance the radiopacity of the stent.

30. The stent of claim 1, wherein the polymeric coil is translucent.

31. The stent of claim 1, wherein the cylindrical rings include a material therein to enhance the radiopacity of the stent.

32. The stent of claim 1, wherein the cylindrical rings are substantially translucent.

33. The stent of claim 1, wherein the stent may be expanded by force.

34. An intravascular stent, comprising:
a first polymeric coil with a first helix angle; and
a second metallic coil with a second helix angle attached to the first coil.

35. The stent of claim 34, wherein the metallic coil is disposed around the polymeric coil.

36. The stent of claim 34, wherein the polymeric coil is disposed around the metallic coil.

37. The stent of claim 34, wherein the first helix angle is equal to the second helix angle.

38. The stent of claim 34, wherein the first helix angle is equal to the negative value of the second helix angle.

39. The stent of claim 34, wherein the first coil is longitudinally offset from the second coil.

40. The stent of claim 34, wherein the second coil is formed with a plurality of undulations comprising peaks and valleys.

41. The stent of claim 34, wherein the second coil is bonded with the first coil.

42. An intravascular stent, comprising:
a plurality of cylindrical rings having inner surfaces and outer surfaces and aligned along a longitudinal axis of the stent;
a plurality of polymeric wires aligned along a longitudinal axis of the stent and attached to the cylindrical rings.

43. The stent of claim 42, wherein the plurality of wires are disposed along the inner surface of the cylindrical rings.

44. The stent of claim 42, wherein the plurality of wires are disposed along the outer surface of the cylindrical rings.

45. The stent of claim 42, wherein the cylindrical rings have a plurality of undulations comprising peaks and valleys.

46. The stent of claim 42, wherein the plurality of wires are bonded to the cylindrical rings.

47. The stent of claim 42, wherein the cylindrical rings are comprised of a metallic material.

48. The stent of claim 42, wherein the plurality of wires have undulations having peaks and valleys.

49. The stent of claim 42, wherein the plurality of wires comprise two or more wires.

50. An intravascular stent, comprising:
a plurality of metallic cylindrical rings aligned along a longitudinal axis of the stent; and
a plurality of polymeric links connecting the cylindrical rings along the longitudinal axis.

51. The stent of claim 50, wherein the polymeric links are interspersed between adjacent cylindrical rings.

52. The stent of claim 50, wherein a plurality of polymeric links are connected across three adjacent cylindrical rings.

53. The stent of claim 50, wherein the cylindrical rings have undulations including peaks and valleys.

54. The stent of claim 53, wherein the peaks of each cylindrical ring are axially aligned with the valleys of each adjacent cylindrical ring.

55. The stent of claim 54, wherein at least four links are positioned between respective peaks and valleys of a plurality of adjacent cylindrical rings.

56. The stent of claim 53, wherein the peaks of each cylindrical ring are axially aligned with the peaks of each adjacent cylindrical ring.

57. The stent of claim 56, wherein at least two links are positioned between respective peaks of a plurality of adjacent cylindrical rings.

58. The stent of claim 50, wherein the links have proximal ends with a first circumferential width, distal ends with a second circumferential width, and center sections with a third circumferential width.

59. The stent of claim 58, wherein the third circumferential width of the center section is greater than the first circumferential width of the proximal end and greater than the second circumferential width of the distal end of the link.

60. The stent of claim 58, wherein the third circumferential width of the center section is at least two times the first circumferential width of the proximal end and at least two times the second circumferential width of the distal end of the link.

61. The stent of claim 50, wherein at least some of the plurality of links have a first durometer polymer and at least some of the plurality of links have a second durometer polymer.

62. The stent of claim 50, wherein a plurality of polymeric links include links having a durometer in the range of 1A to 99A.

63. The stent of claim 50, wherein a plurality of links are comprised of a first frictional polymer and a plurality of links are comprised of a second frictional polymer.

64. The stent of claim 50, wherein a plurality of links include a plurality of polymers with coefficients of friction ranging from 0.1 to 0.9.

65. The stent of claim 50, wherein at least some of the links have a first thickness polymer and at least some of the links have a second thickness polymer.

66. The stent of claim 50, wherein a plurality of links have a plurality of polymers ranging from 0.001 to 0.010 inch in radial thickness.

67. A method for forming an intravascular stent, comprising:
forming a polymeric coil with an inner surface and an outer surface;
forming a plurality of metallic cylindrical rings having undulations comprising a plurality of peaks and valleys; and
attaching the plurality of rings to the coil.

68. The method of claim 67, wherein attaching the plurality of rings includes attaching the rings to the inner surface of the coil.

69. The method of claim 67, wherein attaching the plurality of rings includes attaching the rings to the outer surface of the coil.

70. A method for forming an intravascular stent, comprising:
forming a first polymeric coil with an inner and outer surface;
forming a second metallic coil having undulations comprising a plurality of peaks and valleys; and
attaching the second metallic coil to the first polymeric coil.

71. The method of claim 70, wherein attaching the second metallic coil includes attaching the metallic coil to the inner surface of the polymeric coil.

72. The method of claim 70, wherein attaching the second metallic coil includes attaching the metallic coil to the outer surface of the polymeric coil.

73. A method for forming an intravascular stent, comprising:
forming a plurality of metallic cylindrical rings having undulations comprising a plurality of peaks and valleys;
forming a plurality of polymeric links; and
attaching the polymeric links between adjacent cylindrical rings.

74. A method for forming an intravascular stent, comprising:
means for forming a polymeric coil with an outer surface;
means for forming a plurality of metallic cylindrical rings having undulations comprising a plurality of peaks and valleys;
means for attaching the cylindrical rings to the outer surface of the coil.

75. The method of claim 74, wherein the means for forming a polymeric coil comprise molding the coil in an injection molding apparatus.

76. The method of claim 74, wherein the means for forming a plurality of metallic cylindrical rings comprise laser cutting the rings.

77. The method of claim 74, wherein the means for attaching the cylindrical rings to the outer surface of the coil comprise bonding the rings to the coil with a bonding agent.

78. A method of crimping a stent with cylindrical rings disposed around a coil section onto a balloon catheter, comprising:
providing a tool for crimping the stent onto the balloon portion of a catheter;
inserting the stent and balloon catheter into the crimping tool; and
crimping the cylindrical rings of the stent;
whereby the stent is removably secured to the balloon portion of the catheter.

79. A method for delivering through and implanting a stent in a body lumen, comprising:
providing a stent comprising a polymeric coil and a plurality of metallic cylindrical rings;
providing a delivery system having a distal end and proximal end;
mounting the stent on the distal end of the delivery system;
advancing the stent through the vascular system to the body lumen;
expanding and implanting the stent; and
removing the delivery system from the body lumen.
